(19) United States
(12) Patent Application Publication
(10) Pub. No.: US 2023/0154590 A1
(43) Pub. Date: May 18, 2023

(54) ELECTRONIC DEVICE THAT PROVIDES RECOMMENDED COLOR INFORMATION AND METHOD OF OPERATING THE SAME

(71) Applicant: Dongwon GU, Suwon-si, Gyeonggi-do (KR)

(72) Inventor: Dongwon GU, Suwon-si, Gyeonggi-do (KR)

(21) Appl. No.: 17/774,024

(22) PCT Filed: Jul. 26, 2021
(86) PCT No.: PCT/KR2021/009636

§ 371 Date: May 3, 2022

(30) Foreign Application Priority Data
Nov. 16, 2020 (KR) 10-2020-0152765

(51) Int. Cl. G16H 20/40; A61B 5/00; A61B 5/103; G16H 40/67

(52) U.S. Cl. CPC G16H 20/40; A61B 5/444; A61B 5/1032; G16H 40/67; A61B 5/0002

(57) ABSTRACT

An electronic device may include: a communication circuit; a display; and a processor, wherein the processor obtains skin information including a plurality of parameters, and provides at least one recommended color information based on at least one parameter of the plurality of parameters included in the skin information.

### TECHNICAL FIELD

The present disclosure relates to an electronic device that provides recommended color information and a method of operating the same, and more particularly, to an electronic device that provides skin tone recommended information and a method of operating the same.

### BACKGROUND ART

In general, a scar refers to a trace remaining after healing a damaged skin, occurs because the damaged dermis may not be restored to a normal shape in a process in which a skin is treated when damage to a deep layer of the dermis has occurred due to surgery or external injury, and has a different shape than a normal skin. In addition, skin deformities such as vitiligo and stretch marks also have a shape different from that of the normal skin.

Recently, as the importance of beauty has been increased day by day, a treatment for improving the scar or the like has also emerged. A conventional treatment method includes a method such as a simple method of applying a drug to the skin surface of the scar or the like, a laser treatment for returning the skin color of the scar or the like, and surgery for excising the skin of the scar or the like.

As an example of the background art of the present disclosure, Korean Patent No. 10-2165289 (published on Oct. 13, 2020) discloses a cosmetic composition for decreasing activity of tyrosinase of a skin for skin whitening. Korean Application No. 10-2165289 discloses that the cosmetic composition is applied to a skin having an uneven skin tone, a melasmic skin, or a hyperpigmented skin, and decreases an appearance of the skin having the uneven skin tone, the melasmic skin, or the hyperpigmented skin.

As another example of the background art of the present disclosure, Korean Patent No. 10-2020-0051573 (published on May 13, 2020) discloses an applicator device that facilitates topical administration of a controllable amount of a pharmaceutical composition to a desired area (e.g., skin, eyes, ears, nose, mouth, anus, or vagina) of a body surface.

### DISCLOSURE

### Technical Problem

A conventional method of applying a drug to a skin surface has a limitation in allowing the drug to reach the dermis of the skin, and thus, has a disadvantage in that a therapeutic effect is low. In addition, a conventional surgical treatment such as a laser treatment and cicatrectomy has a problem that repeated treatment is required, such that skin recovery takes a long time and a treatment cost is high.

Various embodiments of the present disclosure may provide an electronic deice that may measure a skin color of a user with a single touch and semi-permanently or permanently penetrate a raw material corresponding to the measured skin color into a region such as a scar to make the scar similar to a surrounding skin, and a method of operating the same.

### Technical Solution

According to various embodiments, an electronic device may include: a communication circuit; a display; and a processor, wherein the processor obtains skin information including a plurality of parameters, and provides at least one recommended color information based on at least one parameter of the plurality of parameters included in the skin information.

According to various embodiments, a method of operating an electronic device may include: obtaining skin information including a plurality of parameters; and providing at least one recommended color information based on at least one parameter of the plurality of parameters included in the skin information.

### Advantageous Effects

The electronic device according to various embodiments of the present disclosure may provide information on a color corresponding to a skin of a user to improve convenience and accuracy of a product selection of an operator according to the corresponding color.

### DESCRIPTION OF DRAWINGS

In order to more fully understand the drawings mentioned in the detailed description of the present disclosure, a detailed description of each drawing is provided.

FIG. 1 is a diagram illustrating a system that provides recommended color information according to various embodiments.

FIG. 2 is a block diagram of an electronic device according to various embodiments.

FIG. 3 is a flowchart for describing a method of providing recommended color information by an electronic device according to various embodiments.

FIG. 4 is a diagram for describing a method of obtaining skin information by the electronic device according to various embodiments.

FIG. 5 is a diagram for describing a method of obtaining skin information by the electronic device according to various embodiments.

FIG. 6 is a diagram for describing a method of providing recommended color information by the electronic device according to various embodiments.

FIG. 7 is a diagram for describing a method of providing recommended color information by the electronic device according to various embodiments.

FIG. 8 is a flowchart for describing a method of correcting skin information measured according to a user setting of the electronic device according to various embodiments.

FIG. 9 is a diagram for describing a method of obtaining a user setting by the electronic device according to various embodiments.

FIG. 10 is a block diagram illustrating a detailed configuration of the electronic device according to various embodiments.

FIG. 11 is a flowchart for describing a method of operating the electronic device according to various embodiments.

### BEST MODE FOR INVENTION

Hereinafter, an operation principle according to embodiments of the present disclosure will be described in detail with reference to the accompanying drawings. In addition, in describing embodiments of the present disclosure, when it is decided that a detailed description of known functions or components related to the present disclosure may unnecessarily obscure the gist of the present disclosure, the detailed description will be omitted. In addition, the terms used herein are terms defined in consideration of functions in the present disclosure, and may be construed in different ways by intentions of users and operators, customs, or the like. Therefore, definitions of the terms used herein should be interpreted based on contents throughout the present specification and functions corresponding thereto.

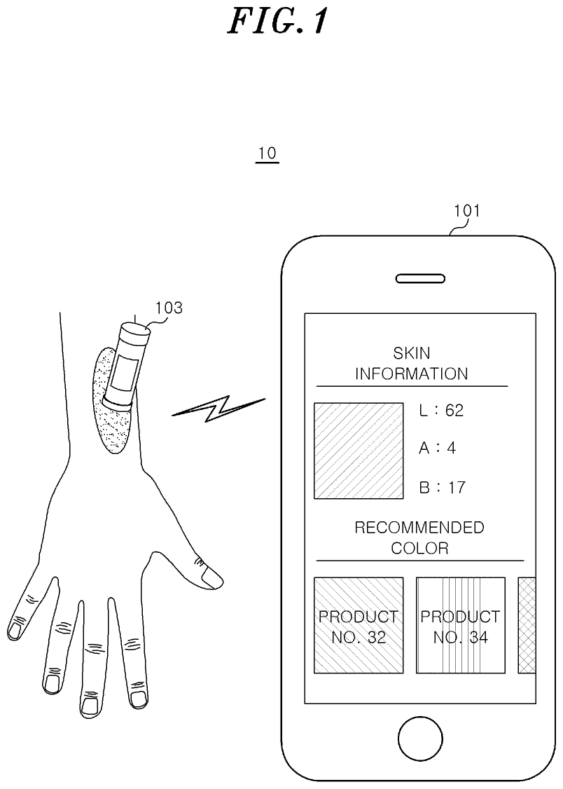

FIG. 1 is a diagram illustrating a system that provides recommended color information according to various embodiments.

Referring to FIG. 1, a system 10 that provides recommended color information may include an electronic device 101 and an external electronic device 103.

According to various embodiments, the electronic device 101 and the external electronic device 103 may transmit and receive data using a short-range communication technology (e.g., near field communication (NFC), Bluetooth, Bluetooth low energy (BLE), wireless fidelity pear to peer (WI-FI P2P), etc.) or a wireless communication technology.

Meanwhile, it has been illustrated in FIG. 1 that the electronic device 101 and the external electronic device 103 directly communicate with each other, but the system 10 that provides recommended color information may further include a server.

For example, the electronic device 101 may communicate with the external electronic device 103 through a server. Specifically, the server may receive information on a measured skin color from the external electronic device 103, identify recommended color information corresponding to the measured skin color, and transmit the recommended color information to the electronic device 101.

According to various embodiments, the external electronic device 103 may measure a skin color.

Specifically, the external electronic device 103 may measure brightness of a skin of a user and saturation corresponding to at least one color through direct and/or indirect contact (e.g., output of vibrations, light of a specific frequency, a high-frequency signal, or the like, to the skin of the user) between the external electronic device 103 and the skin of the user.

For example, the brightness of the skin may be expressed as a parameter value indicating brightness of the skin, and the saturation of the skin may be expressed as a parameter value indicating sharpness corresponding to a red color of the skin and/or a parameter value indicating sharpness corresponding to a yellow color of the skin.

According to an embodiment, the external electronic device 103 is an electronic device capable of measuring skin color information of the user through a camera or the like, but is not limited thereto.

According to various embodiments, the electronic device 101 may receive skin information on the skin color measured by the external electronic device 103 from the external electronic device 103. The electronic device 101 may identify recommended color information corresponding to the measured skin information.

Specifically, the electronic device 101 may identify the recommended color information based on at least one of a brightness parameter, a first saturation parameter, and a second saturation parameter of the measured skin color.

For example, the electronic device 101 may select, as a recommended color, a color having a brightness parameter corresponding to a range in a predefined value, for example, a range in 5%, of a brightness parameter value of the measured skin color.

In this case, a first saturation parameter value or a second saturation parameter value rather than the brightness parameter may be used, and the recommended color may be selected using values of at least two of a plurality of parameters.

According to various embodiments, the electronic device 101 may provide at least the selected recommended color to the user. For example, the electronic device 101 may display at least one selected recommended color on a display, and may output color information (e.g., a color number) to the user as a sound.

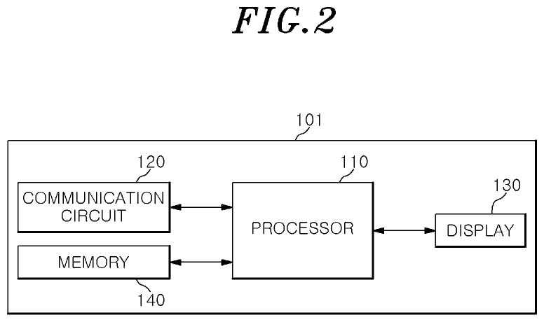

FIG. 2 is a block diagram of an electronic device 101 according to various embodiments.

Referring to FIG. 2, the electronic device 101 may include a processor 110, a communication circuit 120, a display 130, and a memory 140.

In some embodiments, at least one of these components may be omitted in the electronic device 101 or one or more other components may be added to the electronic device 101.

In some embodiments, some of these components may be integrated into one component or one component may be separated into one or more other components.

According to various embodiments, the processor 110 may execute software to control at least one other component of the electronic device 101 connected to the processor 110, and may perform various data processing or operations.

According to various embodiments, the processor 110 may receive the skin information measured by the external electronic device 103 from the external electronic device 103 through the communication circuit 120. Alternatively, the processor 110 may receive the skin information input from the user.

Hereinafter, it will be understood that an operation by the electronic device 101 is performed by an operation of the processor 110.

In addition, the number of processors 110 may be single or plural.

The skin information of the user may include, for example, a brightness parameter indicating the brightness of the skin and a saturation parameter indicating sharpness of a specific color.

Specifically, the brightness parameter may be expressed as a value of 0 or more and 100 or less depending on the brightness of the skin of the user, the first saturation parameter indicating sharpness of a red color may be expressed as a value of 0 or more and 10 or less, and the second saturation parameter indicating sharpness of a yellow color may be expressed as a value of 0 or more and 25 or less.

According to various embodiments, the processor 110 may receive a user setting.

For example, an operation of receiving the user setting may be an operation of determining whether or not to correct a plurality of parameter values included in the skin information of the user and a correction degree of at least one of the plurality of parameter values, in identifying the recommended color information.

For example, a basic mode may refer to a mode in which all of the plurality of parameter values included in the skin information of the user are not corrected, and Mode 1 may be a mode in which a value of a brightness parameter is increased by 10.

According to various embodiments, the processor 110 may identify the recommended color information based on the plurality of parameters included in the skin information.

A plurality of recommended color information may be stored in the memory 140, and each of the plurality of recommended color information may include a brightness parameter, a first saturation parameter, and a second saturation parameter.

For example, the processor 110 may recommend color information having a brightness parameter value within a predefined range with respect to a value of the brightness parameter included in the skin information of the user. As an example, the predefined range may be set to ±5% of the value of the brightness parameter included in the skin information of the user.

Alternatively, the processor 110 may recommend color information having a first saturation parameter value within a predefined range with respect to a first saturation parameter value included in the skin information of the user. As an example, the predefined range may be set to ±5% of the first saturation parameter value included in the skin information of the user.

Alternatively, the processor 110 may recommend color information having a second saturation parameter value within a predefined range with respect to a second saturation parameter value included in the skin information of the user. As an example, the predefined range may be set to ±5% of the second saturation parameter value included in the skin information of the user.

Alternatively, the processor 110 may recommend color information having parameter values with predefined ranges with respect to two or more parameter values selected among the brightness parameter value, the first saturation parameter value, and the second saturation parameter value included in the skin information of the user.

As an example, when the brightness parameter value and the first saturation parameter value are selected, the predefined ranges may be set to ±5% of the brightness parameter value and ±5% of the first saturation parameter value.

As another example, when the brightness parameter value and the second saturation parameter value are selected, the predefined ranges may be set to ±5% of the brightness parameter value and ±5% of the second saturation parameter value.

As still another example, when the first saturation parameter value and the second saturation parameter value are selected, the predefined ranges may be set to ±5% of the first saturation parameter value and ±5% of the second saturation parameter value.

As still another example, when the first saturation parameter value and the second saturation parameter value are selected, the predefined ranges may be set to ±5% of the first saturation parameter value and ±5% of the second saturation parameter value.

As still another example, when the brightness parameter value, the first saturation parameter value, and the second saturation parameter value are selected, the predefined ranges may be set to ±5% of the brightness parameter value, ±5% of the first saturation parameter value, and ±5% of the second saturation parameter value.

The plurality of parameter values included in the skin information described above may refer to parameter values partially corrected according to a mode selected by the user.

According to various embodiments, the processor 110 may provide at least one recommended color information based on the identified recommended color information.

For example, the processor 110 may list and display colors corresponding to at least one recommended color information through the display.

According to various embodiments, the communication circuit 120 may support establishment of a wired communication channel or a wireless communication channel between the electronic device 101 and the external electronic device 103 and execution of communication through the established communication channel.

The communication circuit 120 may include one or more communication processors that operate independently of the processor 110 (e.g., an application processor) and support wired communication or wireless communication. In addition, the communication circuit 120 may include a wireless communication module or a wired communication module.

The wireless communication module and the wired communication module may be integrated into one component or may be implemented as a plurality of components separate from each other. For example, the communication circuit 120 may receive the skin information (or skin color information or skin condition information) measured by the external electronic device 103, from the external electronic device 103 or the server.

According to various embodiments, the display 130 may visually provide information to the outside (e.g., the user) of the electronic device 101. The display 130 may include, for example, a display, a hologram device, or a projector and a control circuit for controlling such a device.

According to an embodiment, the display 130 may include a touch sensor configured to sense a touch or a pressure sensor configured to measure strength of a force generated by the touch to receive a user input.

For example, the display 130 may display recommended color information including information on a predefined number of colors. The recommended color information may include, for example, information on five colors.

Alternatively, the display 130 may receive information on the skin of the user from the user. For example, the display 130 may receive at least one of a brightness value, a first saturation value, and a second saturation value corresponding to the skin of the user.

According to various embodiments, the memory 140 may store various data used by at least one component of the electronic device 101. The data may include, for example, input data or output data on software and instructions related to the software.

For example, the memory 130 may store information on a plurality of recommended colors. For example, the information on the plurality of recommended colors may include a brightness parameter, a first saturation parameter, and a second saturation parameter corresponding to each of the plurality of recommended colors.

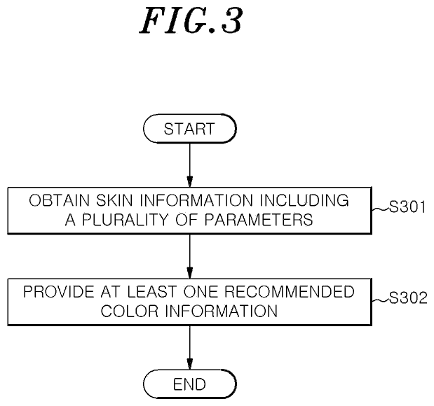

FIG. 3 is a flowchart for describing a method of providing recommended color information by an electronic device according to various embodiments.

The order of respective operations in FIG. 3 is not limited, and another operation may be further performed between two adjacent operations. In addition, at least some of the operations of FIG. 3 may be omitted, and the above description may be equally applied to the entire flowchart of the present disclosure.

In the present disclosure, an expression “the electronic device 101 performs a specific operation” may mean that the processor 110 of the electronic device 101 performs the specific operation or the processor 110 controls other hardware to perform the specific operation.

Alternatively, an expression “the electronic device 101 performs a specific operation” may mean that the processor 110 or other hardware performs the specific operation as the instructions stored in the memory 140 are executed, and may mean that instructions for causing a specific operation to be performed are stored in the memory 140.

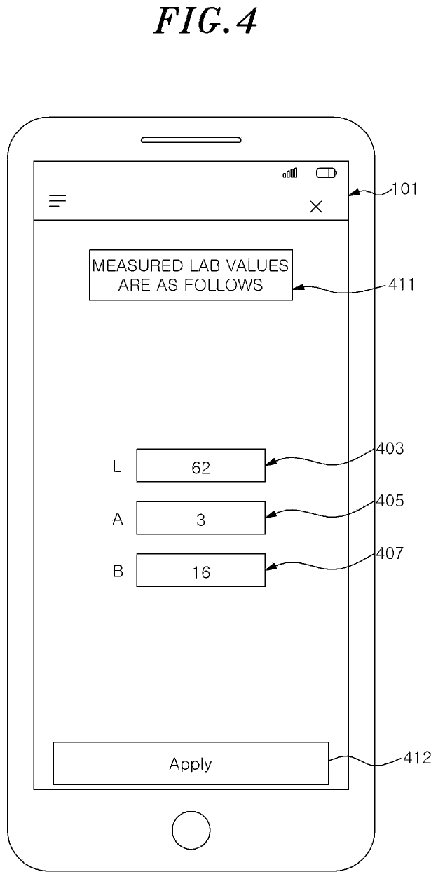

An embodiment of FIG. 3 will be described in more detail with reference to FIGS. 4 to 7.

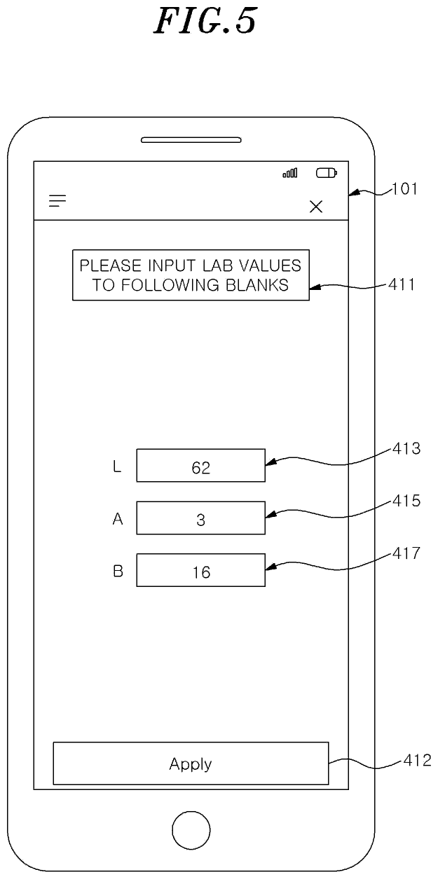

FIGS. 4 and 5 are diagrams for describing a method of obtaining skin information by the electronic device according to various embodiments.

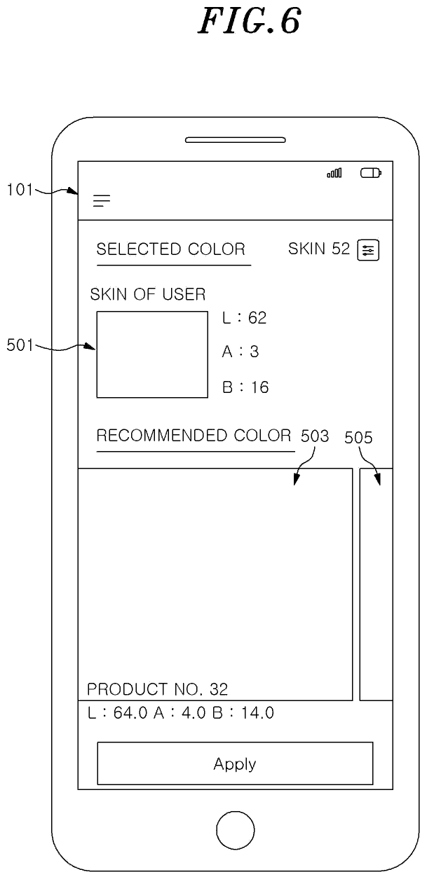

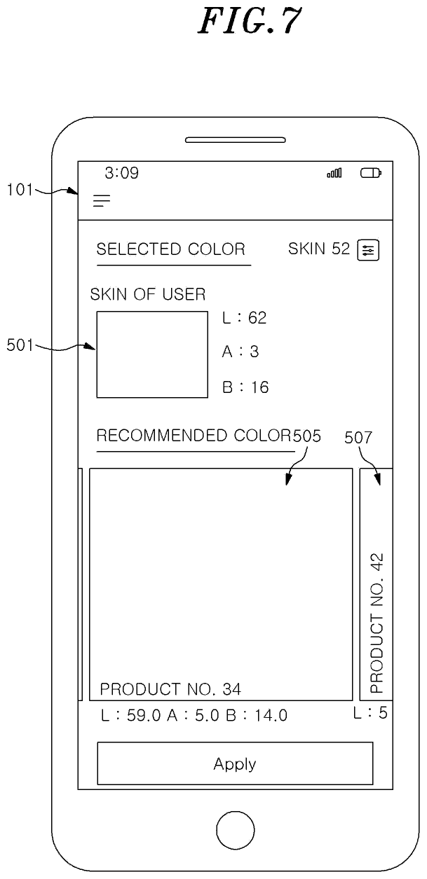

FIGS. 6 and 7 are diagrams for describing a method of providing recommended color information by the electronic device according to various embodiments.

According to various embodiments, the processor 110 may obtain skin information including a plurality of parameters in operation S301.

For example, the skin information may be information on a skin color of the user, and may include a plurality of parameters for indicating the skin color. Specifically, the plurality of parameters may include a brightness parameter indicating brightness of the skin of the user, and for example, the brightness parameter may be expressed as a value of 0 or more to 100 or less according to the brightness.

In addition, the plurality of parameters may include a saturation parameter indicating sharpness of the skin of the user, and the saturation parameter may include a plurality of parameters according to colors.

For example, the saturation parameter may include a first saturation parameter indicating sharpness of a red color and a second saturation parameter indicating sharpness of yellow in a skin color of the user.

In this case, the first saturation parameter may indicate a value of 0 or more to 10 or less according to the sharpness of the red color. The second saturation parameter may indicate a value of 0 or more to 25 or less according to the sharpness of the yellow color.

For example, the processor 110 may receive the skin information of the user measured by the external electronic device 103 from the external electronic device 103. In addition, the processor 110 may display the skin information of the user received from the external electronic device 103.

For example, as illustrated in FIG. 4, the processor 110 may display a guide phrase such as “Measured LAB values are as follows” 411 and values of measured parameters.

For example, the processor 110 may display a value 62 403 of a parameter L indicating brightness of the skin of the user, a value 3 405 of a parameter A indicating sharpness of a red color of the skin, and a value 16 407 of a parameter B indicating sharpness of a yellow color of the skin.

Here, when Apply item 412 is touched in a state in which all of the values L, A, and B are input, a screen may be switched to a screen of FIG. 6 or FIG. 7.

As another example, the processor 110 may receive the skin information input from the user.

For example, as illustrated in FIG. 5, the processor 110 may display a guide phrase for requesting the user to perform an input, such as “Please input LAB values in the following blanks” 411.

In addition, the processor 110 may display a value 62 413 of a parameter L indicating brightness of the skin of the user, a value 3 415 of a parameter A indicating sharpness of a red color of the skin, and a value 16 417 of a parameter B indicating sharpness of a yellow color of the skin input from the user.

Here, when Apply item 412 is touched in a state in which all of the values L, A, and B are input, a screen may be switched to a screen of FIG. 6 or FIG. 7.

According to various embodiments, the processor 110 may provide at least one recommended color information in operation S302. Specifically, the processor 110 may identify recommended color information including parameters similar to the plurality of parameters included in the obtained skin information of the user.

For example, the processor 110 may identify recommended color information including a parameter value similar to at least one of a value of a brightness parameter, a value of a first saturation parameter, and a value of a second saturation parameter among the plurality of parameters.

Alternately, the processor 110 may identify recommended color information including a brightness parameter value similar to the brightness parameter value, recommended color information including a first saturation parameter value similar to the first saturation parameter value, recommended color information including a second saturation parameter value similar to the second saturation parameter value, and/or recommended color information including parameter values similar to two or more parameter values, and provide a list including all of the respective recommended color information.

In this case, the phrase “parameter value similar to the parameter value” may refer to a case where a difference between the parameter values is smaller than a preset value, and when the number of recommended colors including the similar parameters is plural, a list including all of the plurality of recommended colors may be provided.

For example, as illustrated in FIGS. 6 and 7, a brightness parameter value included in skin information 501 of the user may be 62, a first saturation parameter value included in the skin information 501 of the user may be 3, and a second saturation parameter value included in the skin information 501 of the user may be 16.

In this case, for example, the processor 110 may identify a color having a brightness parameter value (that is, a brightness parameter value of 58.9 to 65.1) corresponding to an error range of 3.1, which is 5% of the brightness parameter value 62, as the recommended color.

For example, the processor 110 may recommend colors for five products including a color 503 for Product No. 32 having a brightness parameter L value of 64.0 and a color 505 for Product No. 34 having a brightness parameter L value of 59.0 among the plurality of recommended colors. For example, the plurality of recommended colors may include 52 recommended colors, and respective products (Product Nos. 1 to 52) corresponding to the 52 recommended colors may be manufactured. Here, the product may be a powder or liquid-phase color tone component.

As an example, at least one of the colors for the five products may be displayed on one area 503 of the display 130. In this case, when an input for touching and dragging one area 503 in one direction (e.g., a left direction) is received, colors for other products may be sequentially displayed in the one direction.

In this case, the colors for the five products may be arranged at a predetermined distance.

In addition, the colors for the five products may be arranged according to suitability. In this case, the colors for the five products may be arranged in the one direction from the color for the most suitable product. Here, the suitability may be set higher as a brightness value, a first saturation value, and a second saturation value of a product (or color of a product) become closer to the obtained brightness value, first saturation value, and second saturation value of the skin.

In addition, although not illustrated in FIGS. 6 and 7, the processor 110 may identify colors having similar parameter values to the first saturation parameter value 3 and/or the second saturation parameter value 16 and display a color 503 for Product No. 35 and a color 505 for Product No. 34 together.

Meanwhile, as described above, the processor 110 may recommend the color information by combining two or more of the brightness parameter, the first saturation parameter, and the second saturation parameter with each other.

Specifically, the processor 110 may recommend the color information having parameter values with predefined ranges with respect to two or more parameter values selected among the brightness parameter value, the first saturation parameter value, and the second saturation parameter value included in the skin information of the user.

As an example, when the brightness parameter value and the first saturation parameter value are selected, the predefined ranges may be set to ±5% of the brightness parameter value and ±5% of the first saturation parameter value.

As another example, when the brightness parameter value and the second saturation parameter value are selected, the predefined ranges may be set to ±5% of the brightness parameter value and ±5% of the second saturation parameter value.

As still another example, when the first saturation parameter value and the second saturation parameter value are selected, the predefined ranges may be set to ±5% of the first saturation parameter value and ±5% of the second saturation parameter value.

As still another example, when the first saturation parameter value and the second saturation parameter value are selected, the predefined ranges may be set to ±5% of the first saturation parameter value and ±5% of the second saturation parameter value.

As still another example, when the brightness parameter value, the first saturation parameter value, and the second saturation parameter value are selected, the predefined ranges may be set to ±5% of the brightness parameter value, ±5% of the first saturation parameter value, and ±5% of the second saturation parameter value.

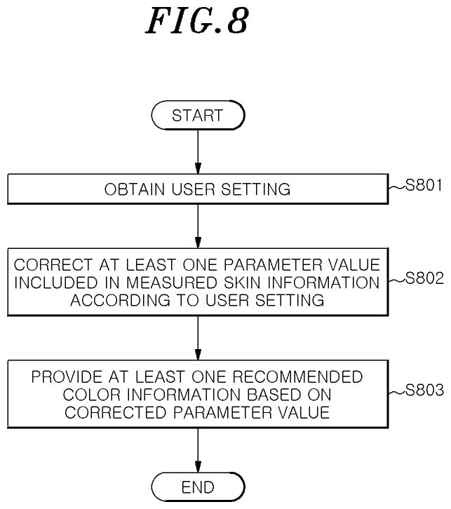

FIG. 8 is a flowchart for describing a method of correcting skin information measured according to a user setting of the electronic device according to various embodiments.

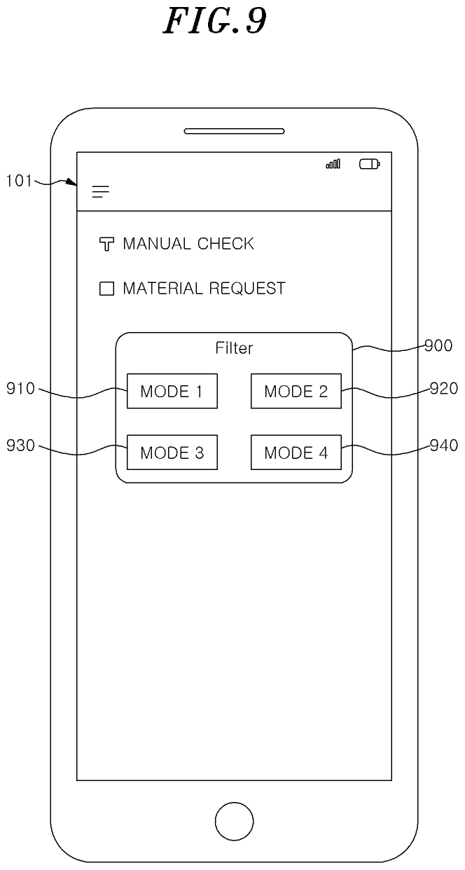

An embodiment of FIG. 8 will be described in more detail with reference to FIG. 9. FIG. 9 is a diagram for describing a method of obtaining a user setting by the electronic device according to various embodiments.

According to various embodiments, the electronic device 101 may obtain a user setting in operation S801.

For example, an operation of receiving the user setting may be an operation of determining whether or not to correct a plurality of parameter values included in the skin information of the user and determining a correction degree of at least one of the plurality of parameter values, before identifying the recommended color information.

Since the correction degree of the parameter value is different depending on a skin condition (e.g., a type of scar) of the user, this may mean setting the correction level of the parameter value according to the skin condition.

For example, as illustrated in FIG. 9, the processor 110 may display a guide window 900 for obtaining the user setting.

The processor 110 may obtain an input for selecting one item among Mode 1 910, Mode 2 920, Mode 3 930, and Mode 4 940 included in the guide window 900.

For example, Mode 1 910 may be a “basic” mode in which the plurality of parameter values included in the skin information of the user are not corrected. Mode 2 920 may be a mode corresponding to stretch marks on a flat skin. Mode 3 930 may be a mode corresponding to corrugated stretch marks. In addition, Mode 4 940 may be a mode corresponding to vitiligo and/or depigmentation.

According to various embodiments, the processor 110 may correct at least one parameter value included in the measured skin information according to the user setting, in operation S802.

For example, when Mode 1 910 corresponding to the basic mode is selected, the processor 110 may maintain the plurality of parameters included in the measured skin information without correcting the plurality of parameters.

For example, when Mode 2 920 corresponding to the stretch marks of the flat skin is selected, the processor 110 may perform correction for increasing a value of a brightness parameter of the plurality of parameters included in the skin information of the user by 10.

For example, when Mode 3 930 corresponding to the corrugated stretch marks is selected, the processor 110 may perform correction for increasing a value of a brightness parameter of the plurality of parameters included in the skin information of the user by 20.

For example, when Mode 4 940 corresponding to the vitiligo and/or the depigmentation is selected, the processor 110 may perform correction for decreasing a value of a brightness parameter of the plurality of parameters included in the skin information of the user by 10.

Meanwhile, although it has been described that the brightness parameter value is corrected according to each mode described above, it will be apparent that a skin condition corresponding to each mode and a correction value corresponding to the skin condition are not limited thereto.

According to various embodiments, the processor 110 may provide at least one recommended color information based on the corrected parameter value in operation S803.

For example, when Mode 1 910 corresponding to the basic mode is selected, the processor 110 does not correct the plurality of parameter values included in the measured skin information, and identify recommended color information corresponding to the plurality of parameter values. In this case, the processor 110 may provide information on colors most similar to the skin of the user as at least one recommended color information.

When a color brighter than an actual skin color of the user by one level is permeated into the stretch marks of the flat skin, the stretch marks of the flat skin may look similar to the actual skin color of the user.

Accordingly, when Mode 2 920 corresponding to the stretch marks of the flat skin is selected, the processor 110 may provide information on colors brighter than the actual skin color of the user by one level as recommended color information through the correction for increasing the value of the brightness parameter by 10.

For example, when the brightness parameter value among the plurality of parameters included in the measured skin information is 62, the first saturation parameter value among the plurality of parameters included in the measured skin information is 3, the second saturation parameter value among the plurality of parameters included in the measured skin information is 16, and the user setting is selected as Mode 2 920, the processor 110 may correct the brightness parameter value to 72, and provide at least one recommended color information based on the fact that the brightness parameter value is 72, the first saturation parameter value is 3, and the second saturation parameter value is 16.

Since the corrugated stretch marks may look darker than the actual skin color due to shadows, when a color brighter than that of the stretch marks of the flat skin by two levels is permeated into the corrugated stretch marks, the corrugated stretch marks may look similar to the actual skin color of the user.

Accordingly, when Mode 3 930 corresponding to the corrugated stretch marks is selected, the processor 110 may provide information on colors brighter than the actual skin color of the user by two levels as recommended color information through the correction for increasing the value of the brightness parameter by 20.

For example, when the brightness parameter value among the plurality of parameters included in the measured skin information is 62, the first saturation parameter value among the plurality of parameters included in the measured skin information is 3, the second saturation parameter value among the plurality of parameters included in the measured skin information is 16, and the user setting is selected as Mode 3 930, the processor 110 may correct the brightness parameter value to 82, and provide at least one recommended color information based on the fact that the brightness parameter value is 82, the first saturation parameter value is 3, and the second saturation parameter value is 16.

The two levels described above may be defined as a state in which the skin is brighter than the one level described above.

Since the vitiligo and/or the depigmentation may have a color brighter than that of the surrounding skin, when a color slightly darker than the measured skin color of the user is penetrated into the vitiligo and/or the depigmentation, the vitiligo and/or the depigmentation may look similar to the actual skin color of the user.

Accordingly, when Mode 4 940 corresponding to the vitiligo and/or the depigmentation is selected, the processor 110 may provide information on colors darker than the actual skin color of the user by one level as recommended color information through the correction for decreasing the value of brightness parameter by 10.

For example, when the brightness parameter value among the plurality of parameters included in the measured skin information is 62, the first saturation parameter value among the plurality of parameters included in the measured skin information is 3, the second saturation parameter value among the plurality of parameters included in the measured skin information is 16, and the user setting is selected as Mode 4 940, the processor 110 may correct the brightness parameter value to 52, and provide at least one recommended color information based on the fact that the brightness parameter value is 52, the first saturation parameter value is 3, and the second saturation parameter value is 16.

According to various embodiments, the processor 110 may obtain a user input for selecting a material request 750. When the user input for selecting the material request 750 is obtained, the processor 110 may display a guide text inquiring whether to move to an Internet page where the material may be purchased.

Accordingly, the user may receive the recommended color information corresponding to the skin condition of the user only through an operation of selecting a mode without directly correcting the color.

The electronic device 101 according to various embodiments of the present disclosure described above may provide the information on the color corresponding to the skin of the user to improve convenience and accuracy of a product selection of an operator according to the corresponding color.

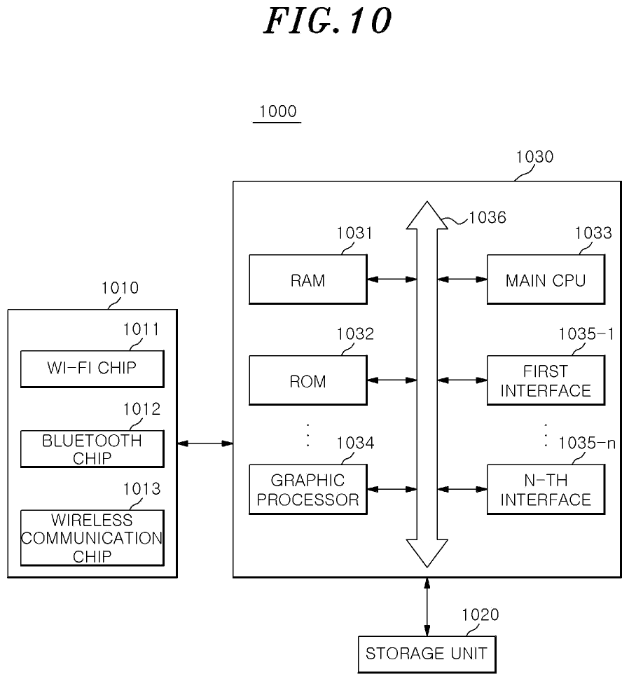

FIG. 10 is a block diagram illustrating a detailed configuration of the electronic device according to various embodiments.

Referring to FIG. 10, the electronic device 1000 includes a communication unit 1010, a storage unit 1020, and a processor 1030.

The communication unit 1000 performs communication. The communication unit 1010 may communicate with an external electronic device through various communication methods such as Bluetooth (BT), wireless fidelity (WI-FI), ZigBee, infrared (IR), and near field communication (NFC).

The storage unit 1020 may store an operating system (O/S) software module for driving the electronic device 1000, data for configuring various user interface (UI) screens provided in a display area, and the like. In addition, the storage unit 1020 may read and write data therefrom and thereto.

In particular, the storage unit 1020 may make a database of color information and product information to which the brightness parameter, the first saturation parameter, and the second saturation parameter are matched, and provide the database to the processor 1030.

The processor 1030 generally controls an operation of the electronic apparatus 1000 using various programs stored in the storage unit 1020.

Specifically, the processor 1030 includes a random access memory (RAM) 1031, a read only memory (ROM) 1032, a main central processing unit (CPU) 1033, a graphic processor 1034, first to n-th interfaces 1035-1 to 1035-n, and a bus 1036.

Here, the RAM 1031, the ROM 1032, the main CPU 1033, the graphic processor 1034, the first to n-th interfaces 1035-1 to 1035-n, and the like, may be connected to each other through the bus 1036.

The first to n-th interfaces 1035-1 to 1035-n are connected to the various components described above. One of the interfaces may be a network interface connected to an external device through a network.

An instruction set for booting a system, or the like, is stored in the ROM 1032. When a turn-on command is input to supply power to the electronic device, the main CPU 1033 copies an 0/S stored in the storage unit 1020 to the RAM 1031 depending on an instruction stored in the ROM 1032, and executes the 0/S to boot the system.

When the booting is completed, the main CPU 1033 copies various stored application programs to the RAM 1031, and executes the application programs copied to the RAM 1031 to perform various operations.

The main CPU 1033 accesses the storage unit 1020 to perform booting using the 0/S stored in the storage 1020. In addition, the main CPU 1033 performs various operations using various programs, contents, data, and the like, stored in the storage unit 1020.

The graphic processor 1034 renders a screen including various objects such as an icon, an image, and a text using a calculator and a renderer.

The processor 1030 may perform the above-described various processes for providing at least one recommended color information based on at least one parameter of the plurality of parameters included in the skin information through the above-described operation.

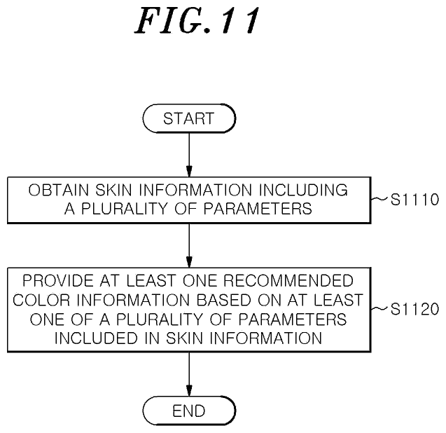

FIG. 11 is a flowchart for describing a method of operating the electronic device according to various embodiments.

Referring to FIG. 11, a method of operating the electronic device may include obtaining skin information including a plurality of parameters (S1110) and providing at least one recommended color information based on at least one of the plurality of parameters included in the skin information (S1120).

Here, the plurality of parameters included in the skin information may include at least one of a brightness parameter indicating brightness of a skin, a first saturation parameter indicating a saturation of the skin corresponding to a first color, or a second saturation indicating a saturation of the skin corresponding to a second color.

In addition, the method of operating the electronic device described above may further include obtaining a user setting and correcting a value of at least one of the brightness parameter, the first saturation parameter, and the second saturation parameter according to the user setting.

In addition, the method of operating the electronic device described above may further include storing a plurality of recommended color information. In this case, each of the plurality of recommended color information may include a brightness parameter, a first saturation parameter, and a second saturation parameter.

In addition, the method of operating the electronic device described above may further include identifying recommended color information having a brightness parameter corresponding to a preset range of a value of the brightness parameter included in the obtained skin information.

In this case, the providing of the at least one recommended color information may include providing the identified recommended color information as the at least one recommended color information.

In addition, the method of operating the electronic device described above may further include identifying recommended color information having a first saturation parameter corresponding to a preset range of a value of the first saturation parameter included in the obtained skin information.

In this case, the providing of the at least one recommended color information may include providing the identified recommended color information as the at least one recommended color information.

In addition, the method of operating the electronic device described above may further include identifying recommended color information having a second saturation parameter corresponding to a preset range of a value of the second saturation parameter included in the obtained skin information.

In this case, the providing of the at least one recommended color information may include providing the identified recommended color information as the at least one recommended color information.

In addition, the method of operating the electronic device described above may further include identifying recommended color information based on a value of the first saturation parameter and a value of the second saturation parameter included in the obtained skin information.

In this case, the providing of the at least one recommended color information may include providing the identified recommended color information as the at least one recommended color information.

The obtaining of the skin information including the plurality of parameters described above may include receiving skin information including a plurality of parameters measured by an external device from the external device.

In addition, the obtaining of the skin information including the plurality of parameters described above may include receiving a value corresponding to each of the plurality of parameters.

In addition, the method of operating the electronic device may include: selecting one of a plurality of modes in which a parameter correction value according to a type of a scar of a skin of a user is preset; obtaining skin information including at least one of a brightness parameter indicating brightness of the skin of the user, a first saturation parameter indicating sharpness of a red color in the skin of the user, and a second saturation parameter indicating sharpness of a yellow color in the skin of the user; correcting at least one of the brightness parameter, the first saturation parameter, and the second saturation parameter based on the selected mode. Here, at least one recommended color information may be provided based on at least one of the corrected brightness parameter, the corrected first saturation parameter, and the corrected second saturation parameter.

Meanwhile, the method of operating the electronic device providing the recommended color information according to the various embodiments of the present disclosure described above may be implemented by program codes that may be executed in a computer and be provided in respective servers or devices so as to be executed by the processor in a state in which it is stored in various non-transitory computer-readable media.

As an example, a non-transitory computer readable medium in which a program performing the obtaining of the skin information including the plurality of parameters and the providing of the at least one recommended color information based on at least one of the plurality of parameters included in the skin information is stored may be provided.

The non-transitory computer readable medium is not a medium that stores data for a while, such as a register, a cache, a memory, or the like, and refers to a medium that semi-permanently stores data and is readable by a device. In detail, the various applications or programs described above may be stored and provided in the non-transitory computer readable medium such as a compact disk (CD), a digital versatile disk (DVD), a hard disk, a Blu-ray disk, a universal serial bus (USB), a memory card, a read only memory (ROM), or the like.

The electronic device according to various embodiments disclosed herein may have various types of devices. The electronic device may include, for example, a portable communication device (e.g., a smart phone), a computer device, a portable multimedia device, a portable medical device, a camera, a wearable device, or a home appliance. The electronic device according to an embodiment of the present specification is not limited to the above-described devices.

It is to be understood that various embodiments of the present specification and terms used in these embodiments do not limit technical features described herein to specific embodiments, and include various modifications, equivalents, and/or substitutions of corresponding embodiments. Throughout the drawings, similar or related components will be denoted by similar reference numerals. A singular form of a noun corresponding to an item may include one or more of the item, unless the context clearly dictates otherwise.

Each of the phrases “A or B”, “at least one of A and B”, “at least one of A or B”, “A, B or C”, “at least one of A, B and C”, and “A, B, or C” used herein may include any one of the items listed together in the corresponding one of the phrases or all possible combinations thereof. The terms “first”, “second”, or the like, may be used only to distinguish one component from the other components, and do not limit the corresponding components in other respects (e.g., importance or a sequence). When any (e.g., first) component is referred to as “coupled” or “connected” to another (e.g., second) component with or without the term “functionally” or “communicatively”, it means that any component may be connected to another component directly (e.g., in a wired manner), wirelessly, or through a third component.

The term “module” used in various embodiments of the present specification may include units implemented by hardware, software, or firmware, and may be used interchangeably with terms such as, for example, logics, logic blocks, components, circuits, or the like. The module may be an integrally configured component or a minimum unit performing one or more functions or a portion thereof. For example, according to an embodiment, the module may be implemented in the form of an application-specific integrated circuit (ASIC).

Various embodiments of the present specification may be implemented by software (e.g., a program) including one or more instructions stored in a storage medium (e.g., an internal memory or an external memory) readable by a machine (e.g., the electronic device). For example, a processor (e.g., the processor 110) of the machine (e.g., the electronic device 101) may invoke at least one of one or more instructions stored in the storage medium from the storage medium and execute the invoked command. This makes it possible for the machine to be operated to perform at least one function according to the invoked at least one instruction. The one or more instructions may include a code generated by a compiler or a code that may be executed by an interpreter. The storage medium readable by the machine may be provided in the form of a non-transitory storage medium. Here, the term ‘non-transitory’ only means that the storage medium is a tangible device and does not a signal (e.g., an electromagnetic wave), and does not distinguish a case where data is semi-permanently stored in the storage medium or a case where data is temporarily stored in the storage medium from each other.

According to an embodiment, the methods according to various embodiments disclosed in the present specification may be included and provided in a computer program product. The computer program product may be traded as a product between a seller and a purchaser. The computer program product may be distributed in the form of a storage medium (e.g., a compact disc read only memory (CD-ROM)) readable by the machine or may be distributed (e.g., downloaded or uploaded) online through an application store (e.g., Play Store™) or directly between two user devices (e.g., smartphones). In a case of the online distribution, at least portions of the computer program product may be at least temporarily stored in the storage medium readable by the machine, such as a memory of a server of a manufacturer, a server of an application store, or a relay server or be temporarily created.

According to various embodiments, each component (e.g., a module or a program) of the components described above may include a single entity or a plurality of entities, and some of the plurality of entities may be separately disposed in another component. According to various embodiments, one or more of the corresponding components described above and their operations may be omitted or one or more other components or their operations may be added. Alternatively or additionally, a plurality of components (e.g., modules or programs) may be integrated into one component. In this case, the integrated component may perform one or more functions of each component of the plurality of components in such a way that these functions are performed by the corresponding component of the plurality of components before the integration. According to various embodiments, operations performed by the modules, the program modules, or the other components may be executed in a sequential manner, a parallel manner, an iterative manner, or a heuristic manner, at least some of the operations may be performed in a different order or be omitted, or one more other operations may be added.

1. An electronic device comprising:
a communication circuit;
a display; and
a processor,
wherein the processor
obtains skin information including a plurality of parameters, and
provides at least one recommended color information based on at least one parameter of the plurality of parameters included in the skin information.

2. The electronic device of claim 1, wherein the plurality of parameters included in the skin information include at least one of a brightness parameter indicating brightness of a skin, a first saturation parameter indicating saturation of the skin corresponding to a first color, or a second saturation parameter indicating saturation of the skin corresponding to a second color.

3. The electronic device of claim 2, wherein the processor
obtains a user setting, and
corrects a value of at least one of the brightness parameter, the first saturation parameter, or the second saturation parameter according to the user setting.

4. The electronic device of claim 2, further comprising a memory storing a plurality of recommended color information,
wherein each of the plurality of recommended color information includes the brightness parameter, the first saturation parameter, and the second saturation parameter.

5. The electronic device of claim 4, wherein the processor
identifies recommended color information having a brightness parameter corresponding to a preset range of a value of the brightness parameter included in the obtained skin information, and
provides the identified recommended color information as the at least one recommended color information.

6. The electronic device of claim 4, wherein the processor
identifies recommended color information having a first saturation parameter corresponding to a preset range of a value of the first saturation parameter included in the obtained skin information, and
provides the identified recommended color information as the at least one recommended color information.

7. The electronic device of claim 4, wherein the processor
identifies recommended color information having a second saturation parameter corresponding to a preset range of a value of the second saturation parameter included in the obtained skin information, and
provides the identified recommended color information as the at least one recommended color information.

8. The electronic device of claim 4, wherein the processor
identifies recommended color information based on a value of the first saturation parameter and a value of the second saturation parameter included in the obtained skin information, and
provides the identified recommended color information as the at least one recommended color information.

9. The electronic device of claim 1, wherein the processor receives the skin information including the plurality of parameters measured by an external electronic device from the external electronic device through the communication circuit.

10. The electronic device of claim 1, wherein the processor receives a value corresponding to each of the plurality of parameters, through the display.

11. A method of operating an electronic device, comprising:
obtaining skin information including a plurality of parameters; and
providing at least one recommended color information based on at least one parameter of the plurality of parameters included in the skin information.

12. The method of operating an electronic device of claim 11, wherein the plurality of parameters included in the skin information include at least one of a brightness parameter indicating brightness of a skin, a first saturation parameter indicating saturation of the skin corresponding to a first color, or a second saturation parameter indicating saturation of the skin corresponding to a second color.

13. The method of operating an electronic device of claim 12, further comprising:
obtaining a user setting; and
correcting a value of at least one of the brightness parameter, the first saturation parameter, or the second saturation parameter according to the user setting.

14. The method of operating an electronic device of claim 12, further comprising storing a plurality of recommended color information,
wherein each of the plurality of recommended color information includes the brightness parameter, the first saturation parameter, and the second saturation parameter.

15. The method of operating an electronic device of claim 14, further comprising identifying recommended color information having a brightness parameter corresponding to a preset range of a value of the brightness parameter included in the obtained skin information,
wherein the providing of the at least one recommended color information includes providing the identified recommended color information as the at least one recommended color information.

16. The method of operating an electronic device of claim 14, further comprising identifying recommended color information having a first saturation parameter corresponding to a preset range of a value of the first saturation parameter included in the obtained skin information,
wherein the providing of the at least one recommended color information includes providing the identified recommended color information as the at least one recommended color information.

17. The method of operating an electronic device of claim 14, further comprising identifying recommended color information having a second saturation parameter corresponding to a preset range of a value of the second saturation parameter included in the obtained skin information,
wherein the providing of the at least one recommended color information includes providing the identified recommended color information as the at least one recommended color information.

18. The method of operating an electronic device of claim 14, further comprising identifying recommended color information based on a value of the first saturation parameter and a value of the second saturation parameter included in the obtained skin information,
wherein the providing of the at least one recommended color information includes providing the identified recommended color information as the at least one recommended color information.

19. The method of operating an electronic device of claim 11, wherein the obtaining of the skin information including the plurality of parameters includes receiving the skin information including the plurality of parameters measured by an external electronic device from the external electronic device.

20. The method of operating an electronic device of claim 11, wherein the obtaining of the skin information including the plurality of parameters includes receiving a value corresponding to each of the plurality of parameters.
